(19) United States
(12) Patent Application Publication
(10) Pub. No.: US 2017/0188638 A1
(43) Pub. Date: Jul. 6, 2017

(54) BRA AND METHOD FOR MANUFACTURING THE BRA

(71) Applicant: Regina Miracle International (Group) Limited, Kwai Chung (HK)

(72) Inventor: Zhenqiang LIU, Kwai Chung (HK)

(21) Appl. No.: 15/397,439

(22) Filed: Jan. 3, 2017

(30) Foreign Application Priority Data
Dec. 31, 2015 (CN) 201511031811.9

(51) Int. Cl. A41C 3/00; A61B 5/0205; A61B 5/00; A41C 3/14; A41C 5/00

(52) U.S. Cl. CPC A41C 3/0064; A41C 3/144; A41C 3/0007; A41C 5/005; A61B 5/4866; A61B 5/0205; A61B 5/6804; A61B 5/02438

(57) ABSTRACT

There is provided a bra comprising a left bra cup and a right bra cup, and the left bra cup and the right bra cup each having an outer layer and an inner layer. The outer layer and the inner layer of a bra cup are attached to each other partially so as to form an opening or cavity between the outer layer and the inner layer, and a monitor configured to collect body data of a wearer of the bra is inserted through the opening and positioned between the outer layer and the inner layer. The monitor is embedded within the bra cup.

### CROSS-REFERENCE TO RELATED APPLICATIONS

This application claims priority to and the benefit of Chinese Patent Application No. 201511031811.9, filed Dec. 31, 2015, the entire disclosure of which is incorporated herein by reference.

### BACKGROUND

Technical Field

The present application generally relates to a bra and a bra cup, and also relates to a method for manufacturing a bra or a bra cup.

Related Art

Currently, a demand for a device that can monitor a person's heart rate is increasing. For example, when exercises or movements are performed, it needs to be ensured that movements are performed under an optimal heart rate state. For the increasing demand, sports bras including a breast heart rate monitor have been developed in markets.

The sports bra is generally designed to be worn in a pullover manner, and has or has no other fixing parts. The bra has cup regions for supporting boobs and a bra band located below the cup regions to keep a position of the bra on a body of a wearer. An existing sports bra is designed to include a conductive fabric located on an inner side surface of the bra band so as to send a heart rate of the wearer to a transmitter located on the bra band or in the bra band. However, because the conductive fabric is disposed on the inner side surface of the bra band and the transmitter is disposed on the bra band, when the bra is designed, aesthetics or comfort of the bra is limited. Generally, women tend to wear sports bras only when they take exercises, and after completing exercises, women return to wear ordinary bras worn in daily lives.

### SUMMARY

The present application aims at providing a bra, a bra cup and a method for manufacturing the bra cup or the bra. The bra cup includes a monitor embedded within the bra cup to collect body data of a wearer of the bra and the resultant bra can be worn as an ordinary bra. The collected data may be wirelessly transmitted to an external device such as a smartphone. The monitor that is embedded within the bra cup may be wirelessly charged.

According to an aspect of the present application, there is provided a bra, the bra comprising a left bra cup and a right bra cup, and the left bra cup and the right bra cup each having an outer layer and an inner layer. The outer layer and the inner layer of at least one of the bra cups are attached to each other partially so as to form an opening or a cavity between the outer layer and the inner layer, and a monitor configured to collect body data of a wearer of the bra is inserted through the opening and/or in the cavity, and is positioned between the outer layer and the inner layer. The monitor is embedded within, or is internal to, the bra cup. The monitor picks up and collects the wearer's body data such as heart beat rate and transmit, preferably wirelessly, an output signal containing the data to an external device such as a smartphone.

In an embodiment, an adhesive layer configured to adhere the outer layer and the inner layer to each other is provided at only a portion between the outer layer and the inner layer, so that the opening or cavity between the outer layer and the inner layer for inserting the monitor is formed at location where there is no adhesive between the outer and the inner layers. The adhesive layer is applied to a portion of an inner surface of the outer layer and/or a portion of an outer surface of the inner layer. Application of heat and pressure to the outer and inner layer causes the outer layer to be adhered to the inner layer at position where the adhesive layer is present. Where the adhesive layer is absent, the inner layer is not attached to the outer layer, and an opening or a cavity is thereby formed between the inner layer and the outer layer. The opening or cavity enables a monitor to be inserted and positioned and embedded within the bra cup between the outer layer and the inner layer.

In an embodiment, the at least one of the bra cups comprises only the left bra cup.

In an embodiment, the body data includes any of, and any combination of, heart rate, consumed calories and step count.

In an embodiment, the inner layer and the outer layer each comprises a fabric layer and a foam layer preferably laminated to each other, and a further foam layer molded onto an inner surface of the outer layer is further provided between the outer layer and the inner layer.

In an embodiment, the monitor comprises a wireless charging coil and a monitoring device for monitoring the wearer's body data.

In an embodiment, the at least one of the bra cups further comprises a first foam pad, the first foam pad having an inner side that is closer to the wearer's body and an outer side that is further away from the wearer's body. The first foam pad comprises a first recess on the inner side, the first recess defining a position of the wireless charging coil in the at least one of the bra cups. The first foam pad further comprises a second recess on the outer side, the second recess defining a position of the monitoring device in the at least one of the bra cups. In another embodiment, the first foam pad can alternatively be replaced by any pad and does not need to comprise foam. For example, such a pad may be a gel pad, a silicone pad, a thermoplastic rubber pad, or a fluid pad.

In an embodiment, the at least one of the bra cups comprises only the left bra cup, and the right bra cup comprises a second foam pad at a position that corresponds to the first foam pad's position in the left bra cup. In another embodiment, the second foam pad alternatively can be replaced by any pad and does not need to comprise foam. For example, such a pad may be a gel pad, a silicone pad, a thermoplastic rubber pad, or a fluid pad.

In an embodiment, the monitor is connected to a first conductive fabric for transmitting the wearer's body data to the monitor.

In an embodiment, the monitor and/or the electronic components associated with the monitor are sealed within plastic film, and an insulating material is situated between the plastic film and the monitor and/or the electronic components associated with the monitor.

In an embodiment, the bra further comprises a left side wing and a right side wing each having an inner piece that is closer to the wearer's body and an outer piece that is further away from the wearer's body. At least one of the side wings comprises a second conductive fabric attached to the inner piece, the second conductive fabric being connected to the first conductive fabric for transmitting the wearer's body data to the monitor.

In an embodiment, the inner piece of the at least one of the side wings comprises two separate parts, and at least a portion of the second conductive fabric is positioned between and attached to each of the two separate parts.

In an embodiment, the inner piece of the at least one of the side wings comprises two separate parts, and a first portion of the second conductive fabric is positioned between and attached to each of the two separate parts, and a second portion of the second conductive fabric is located between the inner piece and the outer piece of the at least one of the side wings.

In an embodiment, the monitor is further connected to a third conductive fabric for transmitting the wearer's body data to the monitor.

In an embodiment, the bra further comprises a center gore between the left bra cup and the right bra cup. The center gore comprises a fourth conductive fabric, the fourth conductive fabric being connected to the third conductive fabric for transmitting the wearer's body data to the monitor.

In another aspect of the present invention, there is provided a method of manufacturing a bra cup. The method comprises the steps of: providing a molded outer layer of the bra cup; providing a molded inner layer of the bra cup; applying an adhesive layer to a portion of an inner surface of the molded outer layer and/or a portion of an outer surface of the molded inner layer; applying heat and pressure to the molded outer layer and the molded inner layer so that the molded outer layer is adhered to the molded inner layer at location where the adhesive layer is present between the molded layer and the molded inner layer, with an opening or cavity formed between the molded outer layer and the molded inner layer at location where the adhesive layer is absent; and inserting a monitor for collecting the body data of a wearer of the bra through the opening and/or in the cavity between the outer layer and the inner layer.

In an embodiment, the monitor comprises a wireless charging coil and a monitoring device for monitoring the wearer's body data and the bra cup comprises a foam pad located between the molded outer layer and the molded inner layer, and the method further comprises the step of: molding the foam pad such that a first recess is formed on an inner side of the foam pad and a second recess is formed on an outer side of the foam pad, the first recess defining a position of the wireless charging coil in the bra cup and the second recess defining a position of the monitoring device in the bra cup. In another embodiment, the foam pad can alternatively be replaced by any pad and does not need to comprise foam. For example, such a pad may be a gel pad, a silicone pad, a thermoplastic rubber pad, or a fluid pad.

In yet another aspect of the present invention, there is provided a bra cup comprising an outer layer and an inner layer. The outer layer and the inner layer of the bra cup are attached to each other partially so as to form an opening and/or a cavity between the outer layer and the inner layer, and a monitor configured to collect body data of a wearer of a garment such as a bra comprising the bra cup is inserted through the opening and/or in the cavity and positioned between the outer layer and the inner layer.

In an embodiment, an adhesive layer configured to adhere the outer layer and the inner layer to each other is provided at only a portion between the outer layer and the inner layer, so that the opening between the outer layer and the inner layer for inserting the monitor is formed at location where there is no adhesive between the outer layer and the inner layer. The adhesive layer is applied to a portion of an inner surface of the outer layer and/or a portion of an outer surface of the inner layer. Application of heat and pressure to the outer and inner layer will cause the outer layer to be adhered to the inner layer at position where the adhesive layer is present. Where the adhesive layer is absent, the inner layer is not attached to the outer layer, and an opening or a cavity is formed between the inner layer and the outer layer. The opening or cavity enables a monitor to be inserted and positioned within the bra cup between the outer layer and the inner layer.

In an embodiment, the bra cup comprising a monitor is a left bra cup of a bra.

In an embodiment, the monitor comprises a wireless charging coil and a monitoring device for monitoring the wearer's body data, and the bra cup further comprises a foam pad, the foam pad having an inner side that is closer to the wearer's body and an outer side that is further away from the wearer's body. The foam pad comprises a first recess on the inner side, the first recess defining a position of the wireless charging coil in the bra cup, and the foam pad further comprises a second recess on the outer side, the second recess defining a position of the monitoring device in the bra cup. In another embodiment, the foam pad can alternatively be replaced by any pad and does not need to comprise foam. For example, such a pad may be a gel pad, a silicone pad, a thermoplastic rubber pad, or a fluid pad.

In an embodiment, the monitor is connected to a first conductive fabric and a second conductive fabric for transmitting the wearer's body data to the monitor. In another embodiment, the monitor is directly connected to one conductive fabric only, although it may be connected indirectly to other conducting component through the one conductive fabric.

In an embodiment, the monitor and/or the electronic components associated with the monitor are sealed within plastic film, and an insulating material is situated between the plastic film and the monitor and/or the electronic components associated with the monitor.

### BRIEF DESCRIPTION OF THE DRAWINGS

In the accompany drawings:

FIG. 1a to FIG. 1c are schematic diagrams of steps of molding bra cups according to an embodiment of the present invention;

FIG. 2a is a schematic diagram of bra cups after trimming according to an embodiment of the present invention;

FIG. 2b shows a sectional view of a bra cup shown in FIG. 2a along a line A-A;

FIG. 3 is a schematic diagram of an electronic monitor according to an embodiment of the present invention;

FIG. 4 is a schematic diagram showing an electronic monitor being sealed between upper and lower plastic films;

FIG. 5 is a schematic diagram showing a monitor formed by cutting along a cut line;

FIG. 6a and FIG. 6b are schematic diagrams of injecting an insulating material into an assembly of monitor sealed within plastic film;

FIG. 7 is a schematic diagram showing molding of a foam pad prior to insertion into a bra cup according to an embodiment of the present invention;

FIG. 8 is a schematic diagram showing molding of another foam pad prior to insertion into another bra cup according to an embodiment of the present invention;

FIG. 9 is a schematic diagram showing the attaching of a monitor to a foam pad according to an embodiment of the present invention;

FIG. 10 is a schematic diagram showing insertion of foam pads into the bra cups according to an embodiment of the present invention;

FIG. 11a and FIG. 11b are respective sectional views of a left bra cup and a right bra cup according to an embodiment of the present invention;

FIG. 12 is a schematic process diagram of manufacturing a side wing or a bra according to an embodiment of the present invention;

FIG. 13a to FIG. 13e are schematic process diagrams of attaching side wings, center gore, and other components to bra cups to form a bra according to an embodiment of the present invention; and

FIG. 14a and FIG. 14b are the front view and bottom view of a bra according to an embodiment of the present invention.

### DETAILED DESCRIPTION

To better understand the present application, all aspects of the present application are described in detail by referring to accompany drawings. It should be understood that the accompany drawings and detailed descriptions are merely descriptions of preferred embodiments of the present application, and do not define the scope of the present application in any manner. Throughout this specification, the term “inner” (as relative to “outer”) is used to indicate a side or surface that is closer to the body of a wearer of a bra, while the term “outer” (as relative to “inner”) is used to indicate a side or surface that is further away from the body of the wearer.

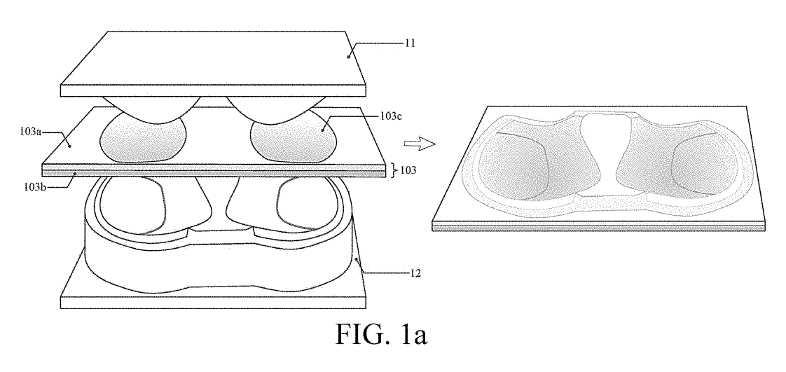

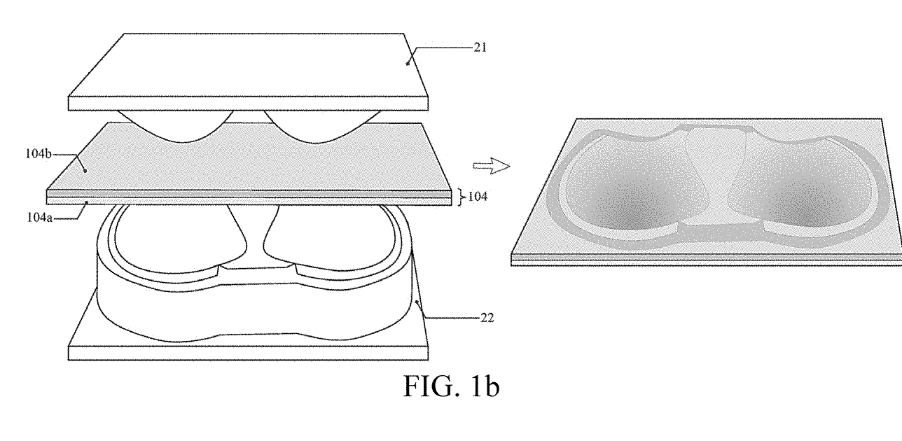

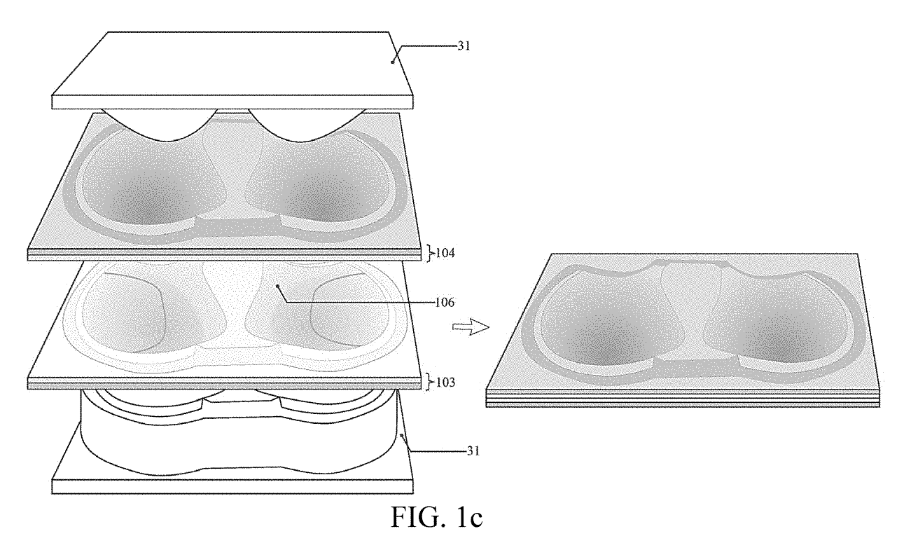

FIG. 1a to FIG. 1c are schematic diagrams of steps of molding bra cups according to an embodiment of the present application. As shown in FIG. 1a, a convex mold portion 11 and a concave mold portion 12 are configured to mold and shape an outer layer 103 of each of two bra cups into a 3D cup shape. The outer layer 103 may comprise, for example, a foam layer 103a laminated with a fabric layer 103b. During molding and shaping of the outer layer 103, a further foam layer 103c may be placed on the side of the foam layer 103a that is not contacting the fabric layer 103b. The further foam layer 103c may extend through the whole surface of the foam layer 103a, or may extend through only a portion of the foam layer 103a. The further foam layer 103c and the fabric-foam laminate 103b, 103a are placed between the convex mold portion 11 and the concave mold portion 12, with the further foam layer 103c being placed on the side of the foam layer 103a that is not in contact with the fabric layer 103b, and are molded together under heat and pressure to form the molded outer layer 103 of the bra cup. This is the first molding step. Adhesive may be applied between the further foam layer 103c and the foam layer 103a to facilitate the affixing of the further foam layer 103c to the foam layer 103a.

With reference to FIG. 1b, a fabric layer 104b laminated with a foam layer 104a is similarly molded between a convex mold portion 21 and a concave mold portion 22 to form a 3D-shaped molded inner layer 104 of each of two bra cups. The mold portions 21 and 22 may be of the same shape as the mold portions 11 and 12 that are used to mold and shape the molded outer layer 103. This is the second molding step.

With reference to FIG. 1c, the molded outer layer 103 and the molded inner layer 104 are placed between a convex mold portion 31 and a concave mold portion 32 such that the foam layer 103a of the molded outer layer 103 and the further foam layer 103c are facing the foam layer 104a of the molded inner layer 104. An adhesive layer 106 is applied between the molded outer layer 103 and the molded inner layer 104. In particular, the adhesive layer 106 is applied to a portion of an inner side of the molded outer layer 103 and/or a portion of an outer side of the molded inner layer 104. The molded outer layer 103 and the molded inner layer 104 are then subject to molding between the convex mold portion 31 and the concave mold portion 32 under heat and pressure. This is the third molding step. After this third step of molding, the portion of the molded outer layer 103 and the molded inner layer 104 to which adhesive layer 106 has been applied would adhere to each other. The portion of the molded outer layer 103 and the molded inner layer 104 to which the adhesive layer 106 has not been applied, in other words the portion where the adhesive layer 106 is absent, would form an opening 108 and/or a cavity, since the molded outer layer 103 and the molded inner layer 104 are not adhered to each other at that location. The opening 108 and/or cavity so formed would allow a monitor to be subsequently inserted between the molded outer layer 103 and the molded inner layer 104. The portion where the adhesive layer 106 is absent preferably includes at least a major portion of the lower region of the bra cup. The portion where the adhesive layer 106 is present, or to which the adhesive layer 106 is applied, preferably includes the neckline region and the region around the side or underarm edge of the bra cup.

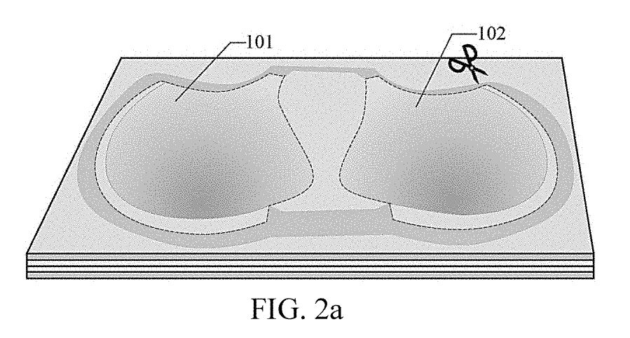

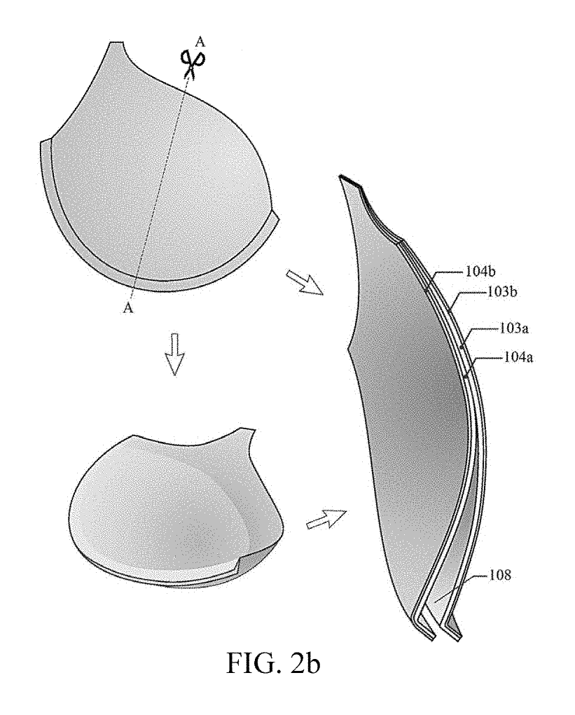

FIG. 2a shows a shape of two bra cups after the aforementioned three molding steps have completed. After molding has completed, the left bra cup 101 and the right bra cup 102 are trimmed from the molded assembly, as illustrated in FIG. 2a. A bra cup so formed, for example the right bra cup, is illustrated in FIG. 2b. In the cross-sectional view along line A-A, the opening or cavity 108 at the lower region of the bra cup is illustrated. At the neckline region and the underarm region, however, it can be seen that the outer layer 103 and the inner layer 104 are attached to each other.

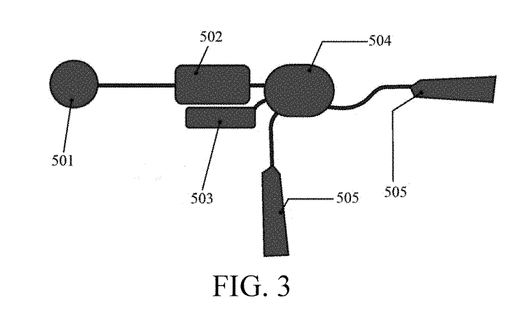

In order to enable monitoring of the body data of a wearer of an ordinary bra, a monitor 50 such as that as shown in FIG. 3 may be installed, hidden or embedded in a bra cup. The monitor 50 typically includes a wireless charging coil 501, a wireless charging printed circuit board assembly (PCBA) 502, a battery 503, and a monitoring assembly 504. The wireless charging coil 501 may be configured to perform wireless charging on the battery 503 of the monitor 50. The monitoring assembly 504 is also connected to at least one, preferably two, conductive fabric 505 in order to transmit data related to the body of the wearer to the monitoring assembly 504. The wireless charging printed circuit board assembly (PCBA) 502 is configured to process data received by the monitoring assembly 504. The data may include, for example, the wearer's heart rate, consumed calories, and step count, etc.

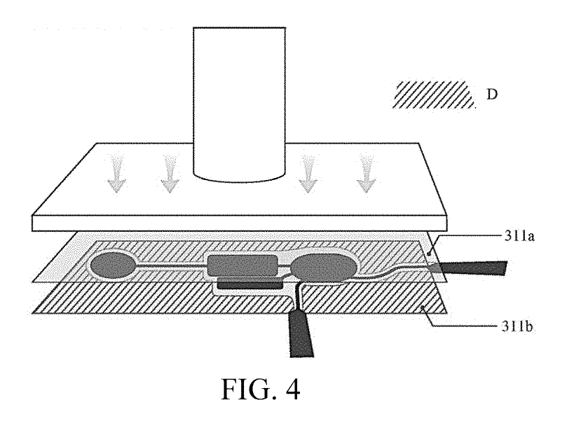

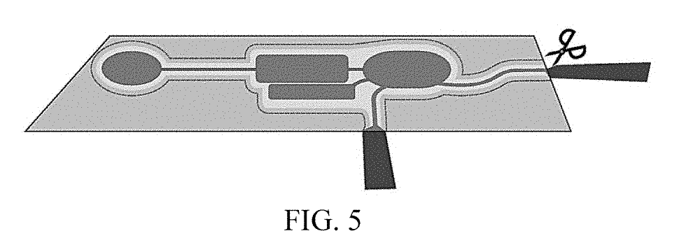

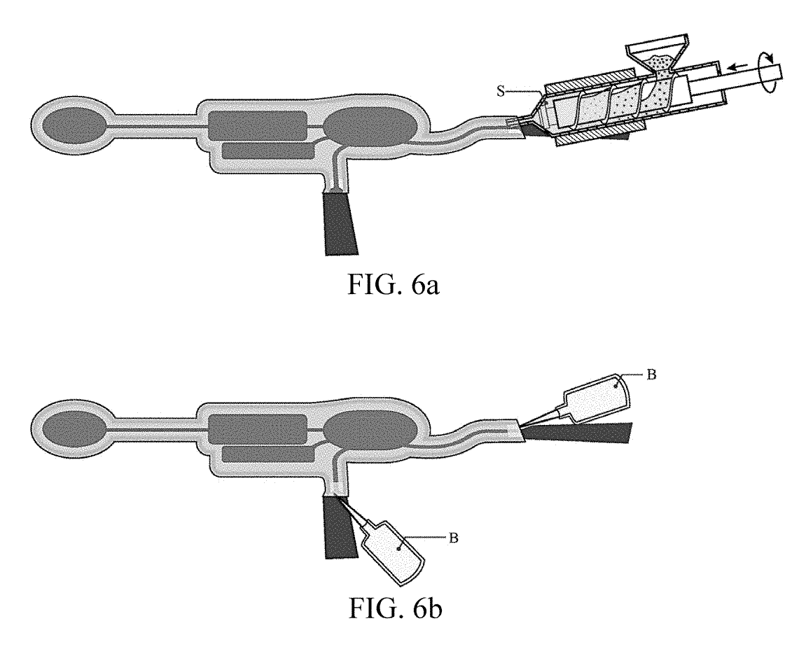

The monitor 50 is described with reference to FIG. 4 and FIG. 5. As illustrated in FIG. 4, the electronic components of or associated with the monitor 50 are placed between two covering plastic films 311a and 311b and the plastic films are sealed to each other, at the sealing zone labelled by D in FIG. 4, by means of high frequency sealing. In one embodiment, a suitable gap is reserved between the electronic components and the plastic films, as represented by the non-sealing zone in FIG. 4. After sealing, the monitor 50 encased within plastic film is trimmed, as illustrated in FIG. 5. In order to provide or enhance insulation between the electronic components and a human body, an insulating material S such as a silicone is injected into the gap between the electronic components and the plastic film, as illustrated in FIG. 6a. The injected insulating material, such as silicone, may also serve to enhance the hand feel of the monitor encased within plastic film. After the insulating material is injected into the gap, the injection opening may be sealed, for example by adhesive B as illustrated in FIG. 6b, so as to prevent the insulating material from leaking from the gap.

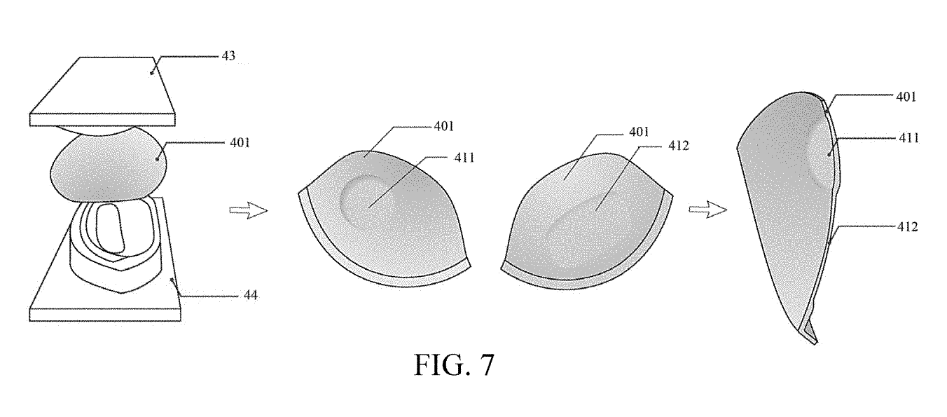

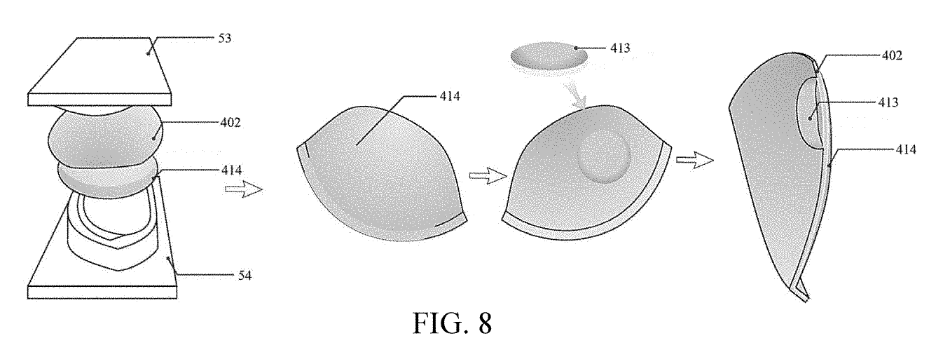

In order to dispose a monitor 50 inside a bra cup such as a left bra cup, a pad such as a foam pad is used as “carrier”. FIG. 7 shows the construction of a foam pad 401 according to an embodiment of the present invention. Referring to FIG. 7, the foam pad 401 comprises a first recess 411 on an inner side of the foam pad, and the first recess 411 defines the position of the wireless charging coil 501 that is to be affixed, for example by adhesive, to the foam pad on its inner side. The foam pad 401 also comprises a second recess 412 on an outer side of the foam pad, and the second recess 412 defines the position of the wireless charging PCBA 502, the battery 503, and the monitoring assembly 504 of the monitor 50 that are to be affixed, for example by adhesive, to the foam pad on its outer side. The recesses are formed by subjecting a foam pad to molding between an upper mold and a lower mold, as illustrated in FIG. 7. In order to ensure consistency of shapes and thickness of the two bra cups of for example a bra, a similar foam pad 402 is provided for the other bra cup such as the right bra cup when the monitor is installed in the left bra cup. For the foam pad 402, similar first and second recesses are formed by molding between an upper mold and a lower mold, as illustrated in FIG. 8, whereby the first recess defines the position of a hard foam pad that corresponds in size, shape and/or hardness to the wireless charging coil, while the second recess defines the position of another hard foam pad that corresponds in size, shape and/or hardness to the other components of the monitor, including the wireless charging PCBA 502, the battery 503, and the monitoring assembly 504 of the monitor 50. In one embodiment, the hard foam pads are affixed to the foam pad 402 by adhesive. In general, the foam pads 401 and 402 consist of foam which is softer than the foam of the hard foam pads that correspond to the various electronic components of the monitor 50.

In an embodiment of the present invention, the foam pad 401 together with the monitor 50 affixed to it is inserted into the opening or cavity 108 of the left bra cup 101, while the “dummy” foam pad 402 is inserted into the opening or cavity 108 of the right bra cup 102. The “dummy” foam pad 402 has hard foam pads affixed to it and the hard foam pads correspond in size, shape and/or hardness to the various components of the monitor 50.

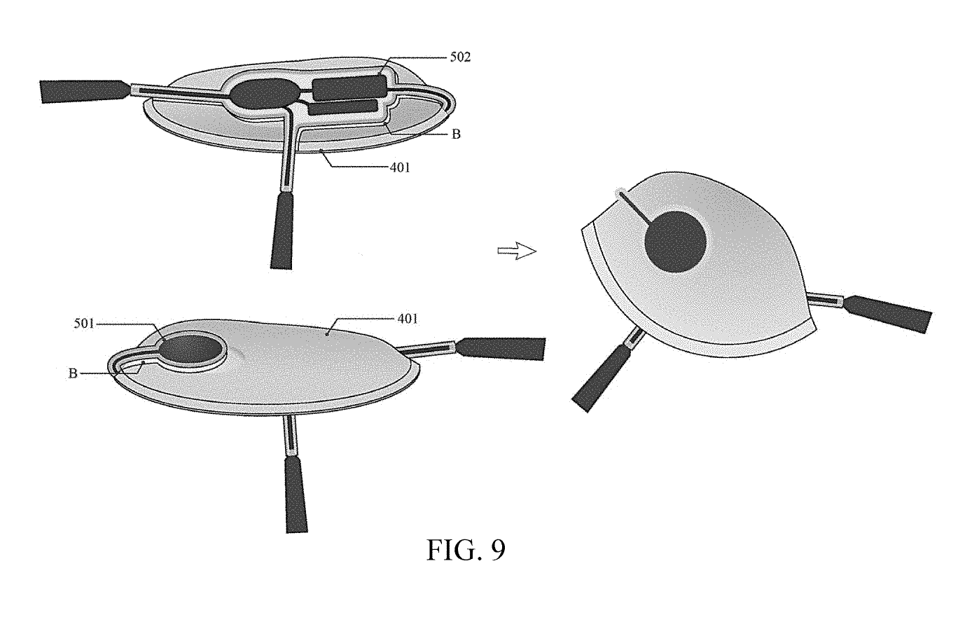

FIG. 9 is a schematic diagram of placing the monitor on a pad, preferably a foam pad, before inserting the pad together with the monitor into a bra cup, i.e. into the cavity and/or through the opening between the outer layer and the inner layer of the bra cup. Referring to FIG. 9, in one embodiment, the wireless charging coil 501 of the monitor 50 is affixed for example by adhesive to an inner side or surface of a foam pad 401 (FIG. 9(b)), while the wireless charging PCBA 502, the battery 503, and the monitoring assembly 504 of the monitor 50 are affixed for example by adhesive to an outer side or surface of the foam pad 401 (FIG. 9(a)). Two conductive fabrics extend from the monitoring assembly 504 and beyond the foam pad 401.

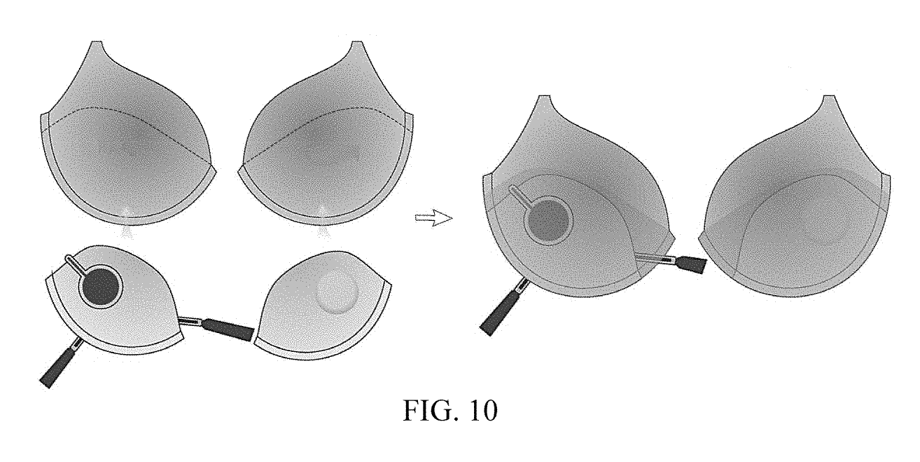

FIG. 10 shows a schematic diagram of two bra cups, which is viewed from the inner side of the bra cups, each provided with a foam pad inserted inside. It can be seen from FIG. 10 that when a foam pad 401 provided with a monitor 50 affixed thereto is inserted into an opening or cavity between the outer layer and the inner layer of a bra cup, for example the left bra cup as shown in FIG. 10, all the electronic components of the monitor 50 are provided inside the bra cup. Hence, a bra that is provided with a monitor 50 inside a bra cup may be washed with water as an ordinary bra. In addition, the monitor 50 collects data related to a human body through at least one conductive fabric 505, preferably two conductive fabrics, extending out of the bra cup. After the insertion of the monitor 50 and the foam pad 401, or the insertion of the “dummy” foam pad 402, the opening 108 may be “closed”, for example by stitching the outer and inner layers 103, 104 together along the lower periphery of the bra cup. This may occur, for example, when an underwire assembly comprising an underwire and an underwire channel is stitched to the bra cup along its lower periphery.

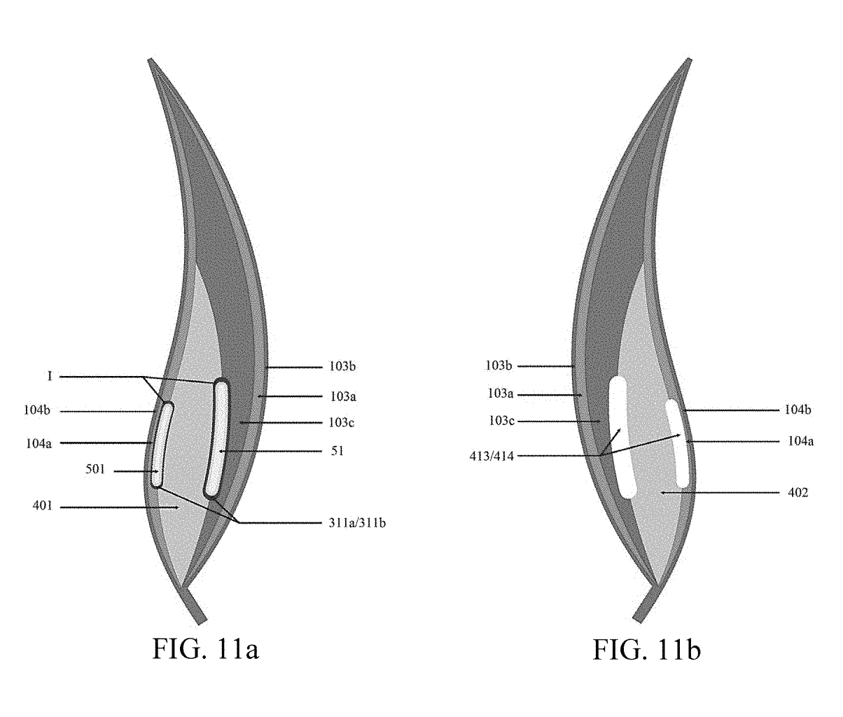

FIG. 11a and FIG. 11b are respective sectional views of a left bra cup 101 and a right bra cup 102 of an embodiment of the present invention. According to an embodiment of the present invention, a monitor 50 is attached to a foam pad 401, and the foam pad 401 together with the monitor 50 is disposed in a cavity 108 within, or embedded within, the left bra cup 101 between the outer layer 103 and the inner layer 104 of the left bra cup. Another foam pad 402, of similar size and shape as the foam pad 401, is similarly disposed in a cavity within, or embedded within, the right bra cup 102. Hard foam pads 413, 414 are provided in the right bra cup 102. These hard foam pads correspond respectively in size, shape and hardness to the monitoring assembly 51 and wireless charging coil 501 in the left bra cup 101, so that a balanced view and feel of the left and right bra cups can be achieved. In an embodiment, the monitor 50 may be a dedicated module for monitoring heart rate. In this case, the monitoring assembly 51 is a heart rate monitoring assembly, and the monitor 50 is disposed on an outer side of a foam pad 401 and is disposed inside the left bra cup 101. A similarly sized and shaped hard foam pad 413 is disposed on an outer side of a foam pad 402 disposed inside the right bra cup 102.

Referring to FIG. 11a, the left bra cup 101 comprises an outer fabric layer 103b, an outer foam layer 103a, an additional outer foam layer 103c, a pad 401, a monitoring assembly 51 disposed on an outer side of the pad 401, a wireless charging coil 501 disposed on an inner side of the pad 401, an inner foam layer 104a, and an inner fabric layer 104b. The pad 401 is preferably a foam pad. The monitoring assembly 51 and the wireless charging coil 501 are encased within plastic film or plastic wrap, and an insulating material such as silicone I fills the gap between the plastic film or wrap and the monitoring assembly and/or the wireless charging coil.

Referring to FIG. 11b, the right bra cup 102 comprises an outer fabric layer 103b, an outer foam layer 103a, an additional outer foam layer 103c, a pad 402, a first hard foam pad 413 disposed on an outer side of the pad 402, a second hard foam pad 414 disposed on an inner side of the pad 402, an inner foam layer 104a, and an inner fabric layer 104b. The position of the first hard foam pad 413 in the right bra cup 102 corresponds to the position of the monitoring assembly 51 in the left bra cup 101, while the position of the second hard foam pad 414 in the right bra cup 102 corresponds to the position of the wireless charging coil 501 in the left bra cup 101. As seen in FIG. 11a and FIG. 11b, the sizes and shapes of the first and second hard foam pads 413, 414 also correspond respectively to the sizes and shapes of the monitoring assembly 51 and the wireless charging coil 501.

Referring to FIG. 11a, it can be seen that the thickness of the inner foam layer 104a of the inner layer 104 of the left bra cup 101 is generally less than the thickness of the outer foam layer 103a of the outer layer 103. The reduced thickness is to enable the wireless charging coil 501 of the monitor 50 to be charged more effectively. The thickness of the inner foam layer 104a of the inner layer 104 is, preferably, less than 8 mm.

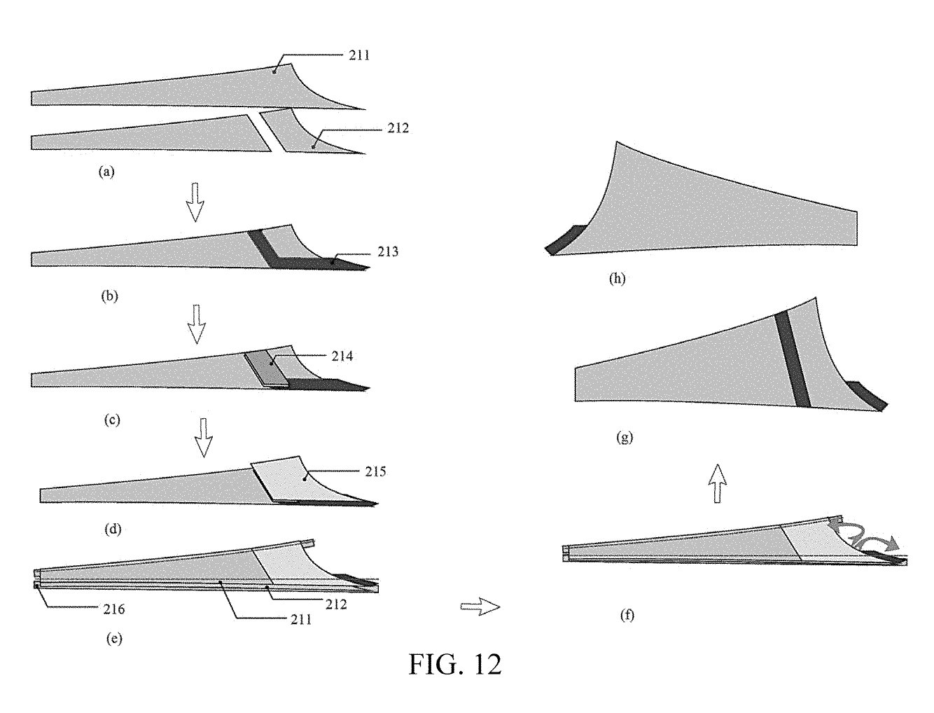

FIG. 12 shows schematic process diagrams of the construction of a side wing according to an embodiment of the present invention. With reference to FIG. 12, a side wing 20 of the present invention preferably includes an outer piece 211 and an inner piece 212. According to an embodiment of the present invention, as illustrated in FIG. 12(a), the inner piece 212 includes two portions 212a and 212b. As shown in FIG. 12(b), a conductive fabric 213 is attached to the portions 212a and 212b on either side of the conductive fabric. The attachment may, for example, be by means of ultrasonic welding or stitching. The conductive fabric 213 is disposed on the inner side of the side wing 20, which is in contact with the wearer's skin, so that the body data of the wearer can be collected and picked up by the conductive fabric 213. With reference to FIG. 12(b), the conductive fabric 213 is generally L-shaped and consists of a first portion that extends in perpendicular to the length of the side wing and a second portion that extends along the length of the side wing. The second portion that extends along the length of the side wing extends beyond the terminating end of the side wing that is to be attached to the bra cup; the extension facilities connection of the conductive fabric to a conductive fabric that extends from the monitor 50 embedded in the bra cup. With reference to FIG. 12(c), the first portion of the conductive fabric 213 that runs perpendicular to the length of the side wing is preferably covered by a covering layer 214. The covering layer 214 may comprise, for example, rubber, foam, silicone, plastic rubber, or a thermoplastic material. The covering layer 214 preferably comprises foam, and the moisture-absorbing property of foam helps retain moisture, for example from sweat, to facilitate better conductivity of the body data. The covering layer 214 is attached to the conductive fabric 213 by means of, for example, adhesive. In addition, with reference to FIG. 12(d), a non-stretchable fabric 215 is preferably disposed to cover the first portion of the conductive fabric 213 and the second portion of the conductive fabric 213 until, but does not extend beyond, the terminating end of the side wing that is to be attached to a bra cup. The non-stretchable fabric 215 is there to enhance the side wing's contact the wearer's body so as to pick up the body signals more efficiently and better. Then, with reference to FIG. 12(e), the outer piece 211 and two rubber strips 216 are also attached, for example by means of stitching, to either side of the inner piece. Subsequently, as illustrated in FIG. 12(f), the whole assembly is turned inside out, i.e. the inside becomes the outside while the outside becomes the inside. As a result of the turning inside out, as illustrated in FIG. 12(g) and FIG. 12(h), the rubber strips 216, the covering layer 214, and the non-stretchable fabric 215 are all located and hidden between the outer piece 211 and the inner piece 212 and not visible. The second portion of the conductive fabric 213 that runs along the length of the side wing is also located and hidden between the outer piece 211 and the inner piece 212 and not visible. The first portion of the conductive fabric 213 that extends perpendicular to the length of the side wing is exposed and visible on the inner side of the side wing, as illustrated in FIG. 12(b), and the part of the second portion of the conductive fabric 213 that extends beyond the terminating end of the side wing that is to be attached to the bra cup is also exposed and visible, as illustrated in FIG. 12(g) and FIG. 12(h). Hence, as illustrated in FIG. 12(h), the outer side of the side wing or of the outer piece 211 is smooth, and as illustrated in FIG. 12(g), the inner side of the side wing or of the inner piece 212 is also substantially smooth, as the first portion of the conductive fabric 213 that extends perpendicular to the length of the side wing basically flushes with the two portions 212a and 212b of the inner piece 212, i.e. the first portion of the conductive fabric 213 is contiguous with and does not overlap the portion 212a on one longitudinal side of the first portion of the conductive fabric 213, and is contiguous with and does not overlap the other portion 212b on the other longitudinal side of the first portion of the conductive fabric 213. Alternatively, although not preferred, the first portion of the conductive fabric 213 that extends perpendicular to the length of the side wing may overlap the inner piece 212.

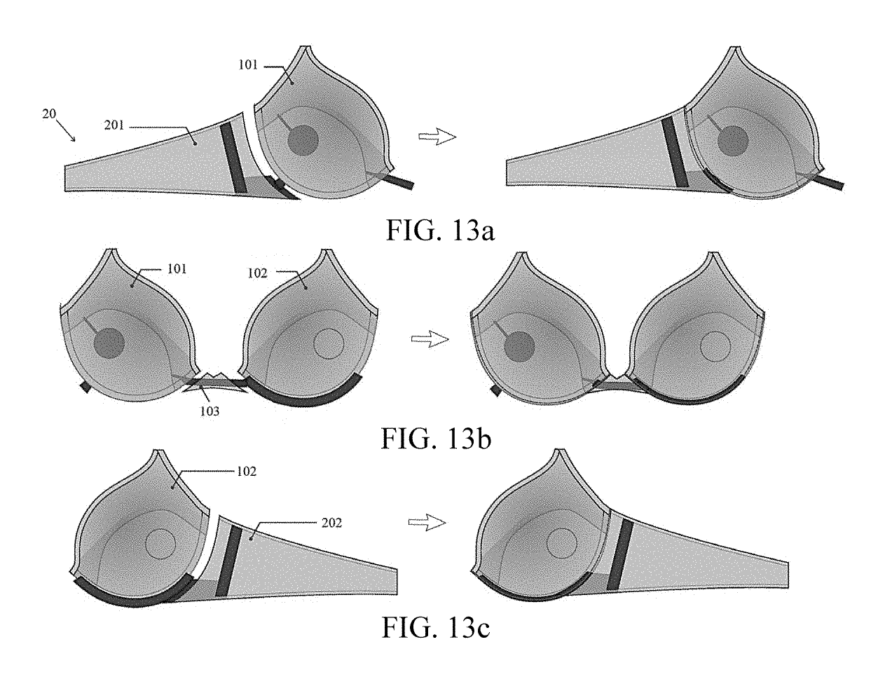

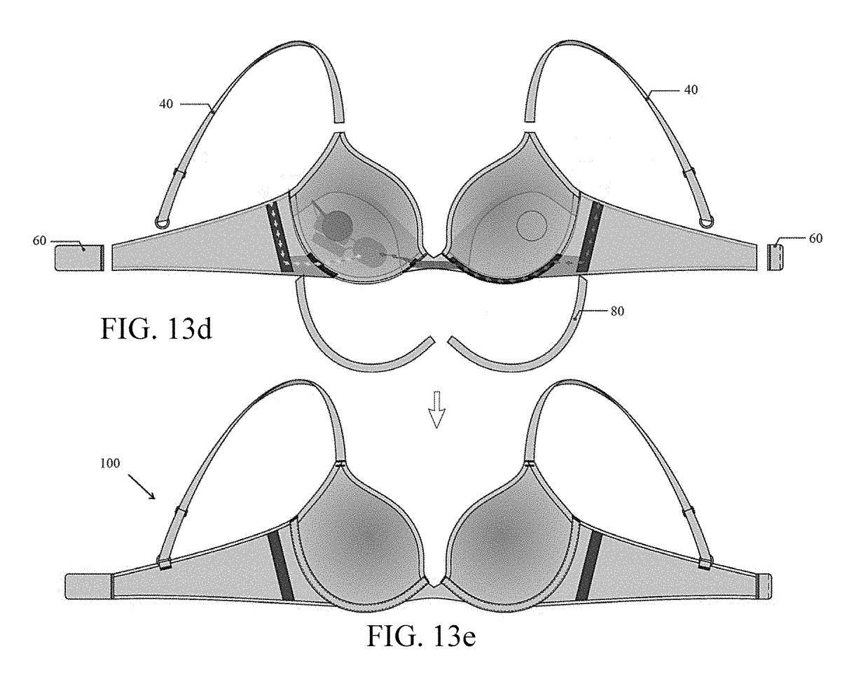

FIG. 13a to FIG. 13e are schematic diagrams illustrating the construction of a bra according to an embodiment of the present invention, as viewed from the inner side of the bra. Thus, FIG. 13a to FIG. 13e show the inner side of a bra of an embodiment of the present invention, and the left and the right sides are construed accordingly, as those sides when viewed from the inner side of the bra. As shown in FIG. 13a, the conductive fabric 213 on the inner side 201 of the left side wing 20 overlaps and is contact with the conductive fabric 505 that is connected to and extends on the left side from the monitoring assembly 504 embedded in the left bra cup 10. Conductive fabric 213 at the side wing and conductive fabric 505 extending from the monitoring assembly 504 are connected to each other through, for example, stitching. Hence, body data collected by means of conductive fabric 213 on the inner side of the side wing which is in contact with the wearer's skin may thereby be transmitted to the monitoring assembly 504 which is connected to the conductive fabric 505. As illustrated in FIG. 13b, a conductive fabric 103 at the center gore is connected, on the left side, to a second conductive fabric that is connected to and extends on the right side from the monitoring assembly 504 embedded in the left bra cup 10, and the conductive fabric 103 at the center gore is also connected, on the right side, to a conductive fabric located at the lower region or the underwire region of the right bra cup. The connection of the conductive fabrics may, for example, be by means of stitching. The conductive fabric at the center gore is preferably located on and attached to the inner side of the center gore. The conductive fabric located at the lower or underwire region of the right bra cup may, for example, be located at and extends along the underwire region, or the region where the underwire channel is normally located. The conductive fabric may preferably be located between the bra cup flange and the underwire channel, so that it is not visible whether on the outer side of the bra or on the inner side of the bra. As a result, body data collected by the conductive fabric at the lower region of the right bra cup may also be transmitted to the monitor 50 embedded in the left bra cup. Also, as shown in FIG. 13c, a conductive fabric is disposed on or attached to the inner side 202 of the right side wing, and this conductive fabric overlaps and is connected to the conductive fabric located at the lower region of the right bra cup 102, with the result that data collected on the inner side of the right side wing 202, which is in contact with the wearer's skin, may be transmitted to the monitor 50 embedded in the left bra cup via the conductive fabric disposed on the inner side of the right side wing, the conductive fabric extending along the lower region of the right bra cup, the conductive fabric extending across the center gore, and the conductive fabric that extends on the right from the monitoring assembly embedded in the left bra cup. All such conductive fabrics form a conducting path for transmission of the body data, and may, for example, be connected by means of stitching.

As shown in FIG. 13d, other components may be attached to form a bra, including straps 40, fasteners 60, and underwire 80. FIG. 13 shows a complete bra according to an embodiment of the present invention as viewed from the inner side, and showing the inner side of the bra, where the conductive fabric at the side wings can be seen.

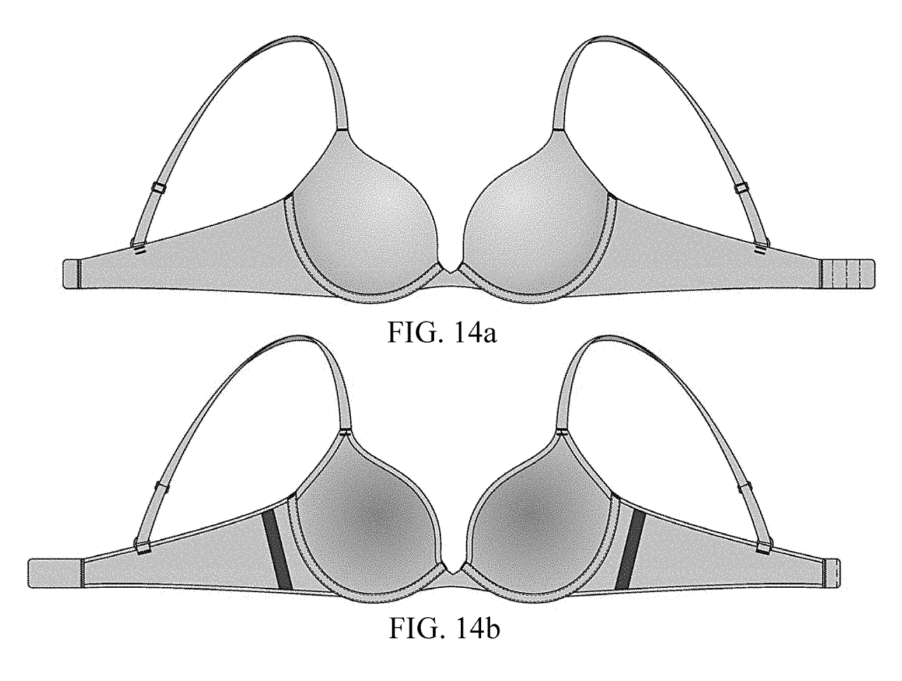

FIG. 14a and FIG. 14b show respectively a front view of an outer side of a bra and the front view of an inner side of a bra of the present invention. It can be seen from the drawings that the monitor 50 is completely disposed inside the bra cup and not exposed or visible. All the electronic components of the monitor 50 are embedded hidden in the bra cup. In addition, because the electronic components of monitor 50 are encased or sealed within plastic film 311, and insulating material is injected and disposed between the electronic components and the plastic film 311, insulation of the electronic components from the outside is ensured, and the bra of the present invention may be washed with water as an ordinary bra.

The exemplary implementation solutions of the present application are described above with reference to accompany drawings. A person skilled in the art shall understand that the implementation solutions are merely examples for description, and are not meant to set limitations. Any modification and equivalent substitution made under teachings and the protection scope of the claims of the present application shall be included in the scope claimed in the present application.

What is claimed is:

1. A bra, comprising a left bra cup and a right bra cup, the left bra cup and the right bra cup each having an outer layer and an inner layer,
wherein the outer layer and the inner layer of at least one of the bra cups are attached to each other partially so as to form an opening between the outer layer and the inner layer, and wherein a monitor configured to collect body data of a wearer of the bra is inserted through the opening and positioned between the outer layer and the inner layer.

2. The bra as claimed in claim 1, wherein an adhesive layer configured to adhere the outer layer and the inner layer to each other is provided at only a portion between the outer layer and the inner layer, so that the opening between the outer layer and the inner layer for inserting the monitor is formed.

3. The bra as claimed in claim 1, wherein the at least one of the bra cups comprises only the left bra cup.

4. The bra as claimed in claim 1, wherein the body data includes heart rate, consumed calories and step count.

5. The bra as claimed in claim 1, wherein the inner layer and the outer layer each comprises a fabric layer and a foam layer, and wherein a further foam layer molded onto an inner surface of the outer layer is further provided between the outer layer and the inner layer.

6. The bra as claimed in claim 1, wherein the monitor comprises
a wireless charging coil; and
a monitoring device for monitoring the wearer's body data.

7. The bra as claimed in claim 6, wherein the at least one of the bra cups further comprises a first foam pad, the first foam pad having an inner side that is closer to the wearer's body and an outer side that is further away from the wearer's body, and wherein the first foam pad comprises a first recess on the inner side, the first recess defining a position of the wireless charging coil in the at least one of the bra cups, and the first foam pad further comprises a second recess on the outer side, the second recess defining a position of the monitoring device in the at least one of the bra cups.

8. The bra as claimed in claim 7, wherein the at least one of the bra cups comprises only the left bra cup, and wherein the right bra cup comprises a second foam pad at a position that corresponds to the first foam pad's position in the left bra cup.

9. The bra as claimed in claim 1, wherein the monitor is connected to a first conductive fabric for transmitting the wearer's body data to the monitor.

10. The bra as claimed in claim 1, wherein the monitor is sealed within plastic film, and an insulating material is situated between the plastic film and the monitor.

11. The bra as claimed in claim 9, wherein the bra further comprises a left side wing and a right side wing each having an inner piece that is closer to the wearer's body and an outer piece that is further away from the wearer's body, at least one of the side wings comprising a second conductive fabric attached to the inner piece, the second conductive fabric being connected to the first conductive fabric for transmitting the wearer's body data to the monitor.

12. The bra as claimed in claim 11, wherein the inner piece of the at least one of the side wings comprises two separate parts, and at least a portion of the second conductive fabric is positioned between and attached to each of the two separate parts.

13. The bra as claimed in claim 11, wherein the inner piece of the at least one of the side wings comprises two separate parts, and a first portion of the second conductive fabric is positioned between and attached to each of the two separate parts, and a second portion of the second conductive fabric is located between the inner piece and the outer piece of the at least one of the side wings.

14. The bra as claimed in claim 9, wherein the monitor is further connected to a third conductive fabric for transmitting the wearer's body data to the monitor.

15. The bra as claimed in claim 14, wherein the bra further comprises a center gore between the left bra cup and the right bra cup, the center gore comprising a fourth conductive fabric, the fourth conductive fabric being connected to the third conductive fabric for transmitting the wearer's body data to the monitor.

16. A method of manufacturing a bra cup, the method comprises the steps of:
providing a molded outer layer of the bra cup;
providing a molded inner layer of the bra cup;
applying an adhesive layer to a portion of an inner surface of the molded outer layer and/or a portion of an outer surface of the molded inner layer;
applying heat and pressure to the molded outer layer and the molded inner layer so that the molded outer layer is adhered to the molded inner layer with an opening formed between the molded outer layer and the molded inner layer at position where the adhesive layer is absent; and
inserting a monitor for collecting the body data of a wearer of the bra between the outer layer and the inner layer through the opening.

17. The method as claimed in claim 16, wherein the monitor comprises a wireless charging coil and a monitoring device for monitoring the wearer's body data and the bra cup comprises a foam pad located between the molded outer layer and the molded inner layer, and wherein the method further comprises the step of:
molding the foam pad such that a first recess is formed on an inner side of the foam pad and a second recess is formed on an outer side of the foam pad, wherein the first recess defines a position of the wireless charging coil in the bra cup and the second recess defines a position of the monitoring device in the bra cup.

18. A bra cup, comprising an outer layer and an inner layer, wherein the outer layer and the inner layer of the bra cup are attached to each other partially so as to form an opening between the outer layer and the inner layer, and wherein a monitor configured to collect body data of a wearer of a garment comprising the bra cup is inserted through the opening and positioned between the outer layer and the inner layer.

19. The bra cup as claimed in claim 18, wherein an adhesive layer configured to adhere the outer layer and the inner layer to each other is provided at only a portion between the outer layer and the inner layer, so that the opening between the outer layer and the inner layer for inserting the monitor is formed.

20. The bra cup as claimed in claim 18, wherein the monitor comprises a wireless charging coil and a monitoring device for monitoring the wearer's body data, and wherein the bra cup further comprises a foam pad, the foam pad having an inner side that is closer to the wearer's body and an outer side that is further away from the wearer's body, and wherein the foam pad comprises a first recess on the inner side, the first recess defining a position of the wireless charging coil in the bra cup, and the foam pad further comprises a second recess on the outer side, the second recess defining a position of the monitoring device in the bra cup.

21. The bra cup as claimed in claim 18, wherein the monitor is connected to a first conductive fabric and a second conductive fabric for transmitting the wearer's body data to the monitor.

22. The bra cup as claimed in claim 18, wherein the monitor is sealed within plastic film, and an insulating material is situated between the plastic film and the monitor.
